(19) United States
(12) Patent Application Publication
(10) Pub. No.: US 2010/0137876 A1
(43) Pub. Date: Jun. 3, 2010

(54) SURGICAL MICROMANIPULATOR TIP, SURGICAL MICROMANIPULATOR AND METHOD FOR PRODUCING A TIP FOR ONE SUCH MICROMANIPULATOR

(76) Inventor: Jean-Marie Andre, Marseille (FR)

Correspondence Address: David W. Highet, VP & Chief IP Counsel;Becton, Dickinson and Company, (Hoffman & Baron), 1 Becton Drive, MC 110, Franklin Lakes, NJ, 07417-1880, US

(21) Appl. No.: 12/517,797

(22) PCT Filed: Dec. 10, 2007
(86) PCT No.: PCT/FR07/02029

§ 371 Date: Feb. 5, 2010

(30) Foreign Application Priority Data
Dec. 11, 2006 (FR) 0610777

(51) Int. Cl. A61F 9/007; B23P 11/00

(52) U.S. Cl. 606/107; 29/428

(57) ABSTRACT

The invention relates to a method for producing a surgical micromanipulator tip including a proximal mounting base, at least a first elbow, and a distal manipulator finger. The elbow is connected to the mounting base and terminates at the free end of the tip. In addition, the mounting base includes at least one distal segment. The first elbow and the manipulator finger together form a tip portion. The tip portion is uniformly tapered by pointing.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to the field of precision instrumentation used in microsurgery, for example in ophthalmic surgery. More precisely it concerns a surgical micromanipulator tip, a surgical micromanipulator equipped with this tip as well as a method for producing such a tip.

2. Description of Related Art Including Information Disclosed Under 37 CFR 1.97 and 37 CFR 1.98

During cataract operations, micromanipulators are used for manipulating crystallines. More precisely, such a manipulator is used during two operating procedures which must be performed with very high precision inside the eye and which are the excision of the natural crystalline to be replaced and then the positioning of the artificial crystalline in its place.

A surgical cataract micromanipulator usually comprises a steel handle and an elbowed tip at one of the ends of this handle. Such a tip as found on present micromanipulators includes a straight portion 5 which includes a proximal mounting base 7, an elbow 4 and a distal manipulation finger 6 connected to said straight portion 5 of this elbow 4. The mounting base 7 is provided to be immobilized in the handle of the micromanipulator while the finger 6 is intended to penetrate into the eye for manipulating the crystalline there. From the mounting base 7 or essentially the remaining portion 9-4-6 of the tip is tapered in the direction of its extremely fine free end.

At present such a tip is produced from a straight steel wire segment which is ground by cylindrical grinding. More precisely, this segment is abraded by two grinding wheels. Between these two grinding wheels it rotates around itself in the opposite direction of the rotational direction of said grinding wheels. Its rotational axis and those of these grinding wheels are parallel to each other. The grinding wheels have a profile that is complementary of the profile in longitudinal section which the grinding operation is meant to give to the segment. To this effect these grinding wheels are dressed with diamonds. The cylindrical grinding of the segment of steel wire hence requires specific grinding wheels that are expensive.

The cost of the present micromanipulators provided with tips of this type is very high already an inconvenience by itself. Furthermore, because of their high cost these micromanipulators cannot be discarded straight after their first use. Thus they cannot be re-used without having previously been cleaned and made aseptic which implies instituting a special organization, beginning with the collection of soiled micromanipulators and ending with their contamination-free storage after their sterilization, as well as additional cost resulting from the mobilization of qualified personnel and an investment in equipment and in the training of such personnel.

The cost of manufacturing ophthalmic micromanipulators by cylindrical grinding constitutes the main obstacle to the routine utilization of such hybrid (plastic and metal) instruments for one-time use.

The objective of the invention is to at least allow a reduction in the cost of using a surgical micromanipulator.

### BRIEF SUMMARY OF THE INVENTION

According to the invention, this goal is achieved because of a surgical micromanipulator tip comprising a proximal mounting base, at least one first elbow and a distal manipulation finger. This first elbow connects to the mounting base and terminates in a free end of the tip. At least one distal segment of the mounting base, the first elbow and the manipulation finger form together a tip portion which tapers in an essentially uniform way over its entire length in the direction of the free end, by featuring a diminishing diameter at an essentially constant rate of linear diminution or by featuring a succession without detachment of several portions. Each portion tapers in an essentially uniform manner over its entire length in the direction of said free end. More precisely, the succession is of at least two frustum-truncated cones, namely a first truncated cone and a second truncated cone the first elbow of which connects a proximal basis and a distal top of the first truncated cone. The micromanipulator tip is mainly noteworthy because it has been tapered by means of pointing.

The objective of the invention is also a manufacturing method for a surgical micromanipulator tip of the type that comprises a proximal mounting base, at least one first elbow and a distal manipulation finger. The first elbow connects to the mounting base and ends in the free end of the tip. At least one distal segment of the mounting base, the first elbow and the manipulation finger form together a tip portion characterized in the uniform tapering of said tip portion, from a straight segment of metallic wire, being achieved by means of pointing.

The tapering of ophthalmic micromanipulator tips by means of pointing requires a non-specific grinding wheel which allows to increase the speed of production and to lower the production costs which makes it possible to manufacture an inexpensive single-use instrument.

### BRIEF DESCRIPTION OF THE SEVERAL VIEWS OF THE DRAWINGS

The invention will be well understood when reading the following description which is given only as an example and which is made while referring to the attached drawings.

FIG. 1 is a schematic side view of an ophthalmic surgical micromanipulator in accordance with the invention.

FIG. 2 is a side elevation view of a tip constitutive of the micromanipulator of FIG. 1 and in accordance with invention.

FIG. 3 is a side elevation view similar to FIG. 2, representing an ophthalmic surgical micromanipulator tip in accordance with a production variance of the invention.

FIG. 4 is a detailed elevation view, at an enlarged scale, showing the free end of the micromanipulator tip shown in FIG. 3.

FIGS. 5, 6, and 7 are schematic views, respectively, of the front, in a section along line A-A of FIG. 5, and from above, illustrating the production method of the tips by means of pointing.

Reference is made to said drawings to describe advantageous examples, although by no means limiting, of producing micromanipulator tips and of the application of the method in accordance with the invention.

### DETAILED DESCRIPTION OF THE INVENTION

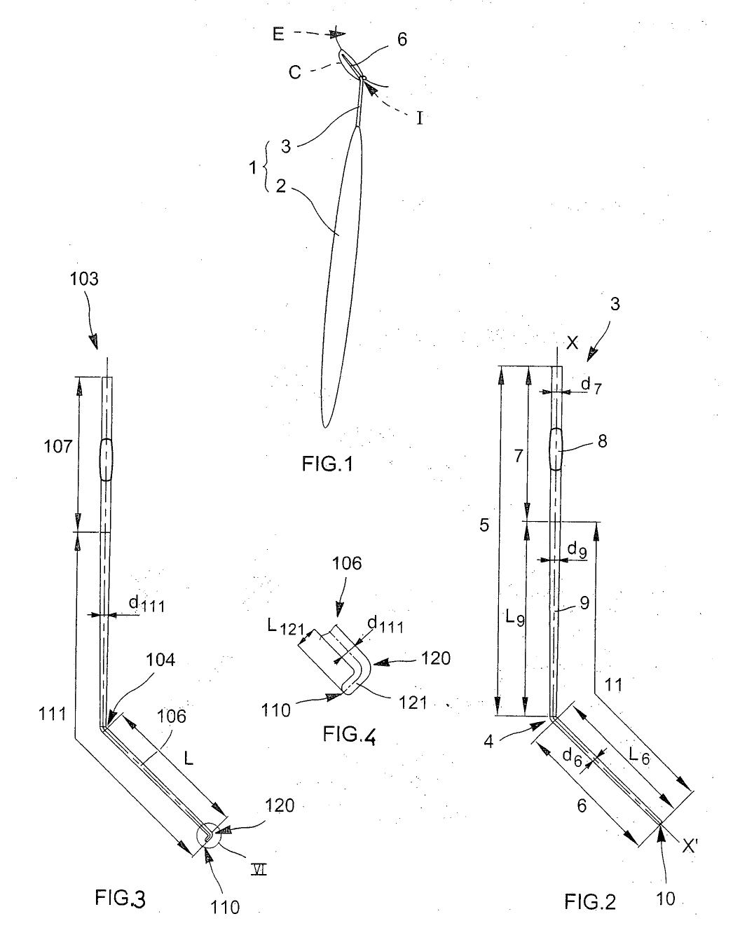

FIG. 1 shows an ophthalmic surgical micromanipulator 1, which includes a handle 2 molded in polymer and a metallic tip 3 held by this handle 2 and preferably made of stainless steel, in particular of inox 302.

As one can see very well on FIG. 2, the tip 3 of the micromanipulator includes an elbow 4 which connects, between them, a proximal mounting base 5 and a distal manipulation finger 6. Contrary to finger 6, a proximal section 7 of the mounting base 5 is intended to be immobilized in the handle 2 and comprises to this effect a flattened part 8. This proximal section 7 is essentially cylindrical except at the level of the flattened part 8. The mounting base 5 also includes a distal section of an overall tapered shape 9 which extends the proximal section 7 up to the elbow 4 and which slims down uniformly over its entire length, in the direction of this elbow 4.

Like the section 9, the finger 6 has the overall shape of a truncated cone. It slims uniformly over its entire length beginning at the elbow 4, in contrast to which it ends in a rounded, free end 10.

The reference 11 designates the assembly formed by the section 9, the elbow 4 and the finger 6, i.e., the section which goes from the free end 10 to the junction of truncated cones 7 and 9 of the mounting base 5. This section 11 slims down over its entire length, in direction of the free end 10, and includes no detachment whatsoever.

On the section 9, the tip 3 presents a circular cross section and its width, equal to its diameter d9, decreases according to a linear diminution rate that is essentially constant towards the free end 10.

On the finger 6, the tip 3 presents a circular cross section and its width, equal to its diameter d6, decreases at a linear diminution rate that is essentially constant towards the free end 10.

The rate of linear diminution of the diameter d9 is at the most equal to that of the diameter d6.

The diameters d9 and d6 may decrease at the same linear diminution rate, in which case the reference 11 designates a portion that is tapering at an essentially uniform manner over its entire length, in the direction of said free end 10.

Preferably the rate of linear diminution of the diameter d9 is inferior to that of the diameter d6 which is the case in the example shown. The reference 11 designates thus a succession without detachment of two truncated cones 6 and 9 of which one has its distal top connected, by the elbow 4, to the base of the other truncated cone 6.

The section 9 has a length L9 advantageously comprised between 10 mm and 20 mm, preferably between 13 mm and 17 mm, for example in the order of 15 mm.

The finger 6a has a length L6 advantageously comprised between 8 mm and 13 mm, preferably between 9 mm and 12 mm, for example in the order of 11 mm.

The proximal section 7 of the mounting base 5 has a diameter d7 advantageously comprised between 0.7 mm and 0.9 mm, preferably between 0.75 mm and 0.85 mm, for example in the order of 0.8 mm.

The sections 7 and 9 of the mounting base 5 have the same diameter at their junction.

Beginning at this junction, the diameter d9 of the section 9 decreases towards the free end 10 by losing advantageously between 0.015 mm to 0.025 mm for each millimeter of length traveled towards the free end 10, preferably between 0.017 mm and 0.023 mm for each millimeter of length traveled towards the free end 10, for example in the order of 0.017 mm for each millimeter of length traveled towards the free end 10.

At the elbow 4, the diameter of the tip 3 is advantageously comprised between 0.48 mm and 0.60 mm, preferably between 0.50 mm and 0.58 mm, for example in the order of 0.56 mm.

Beginning at the elbow 4, the diameter d6 of the finger 6 decreases towards the free end 10 by losing advantageously from 0.015 mm to 0.025 mm for each millimeter of length traveled to the free end 10, preferably from 0.017 mm to 0.023 mm for each millimeter of length traveled to the free end 10, for example in the order of 0.021 mm for each millimeter of length traveled to the free end 10.

At this free end 10, the diameter of the tip 3 is advantageously comprised between 0.18 mm and 0.42 mm, preferably between 0.20 mm and 0.40 mm, for example in the order of 0.38 mm.

According to the invention, the tips 3 are made of metallic segments F which come for example from a reel of steel wire, preferably of Inox 302, and by means of pointing.

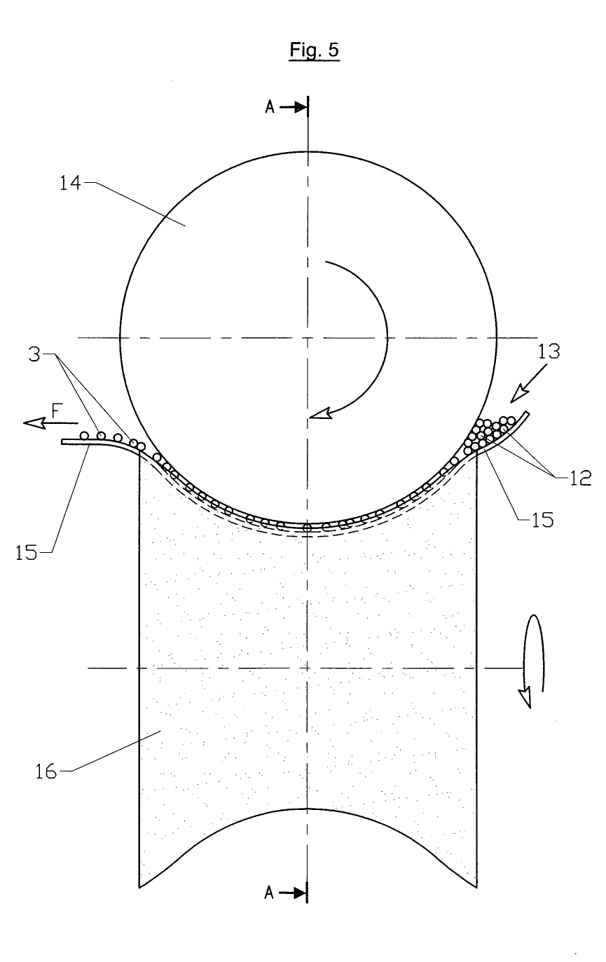

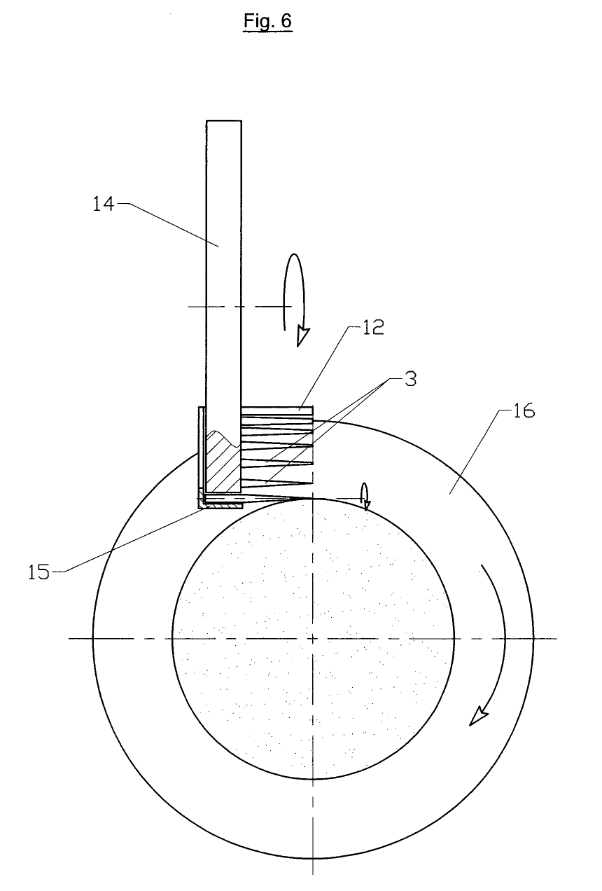

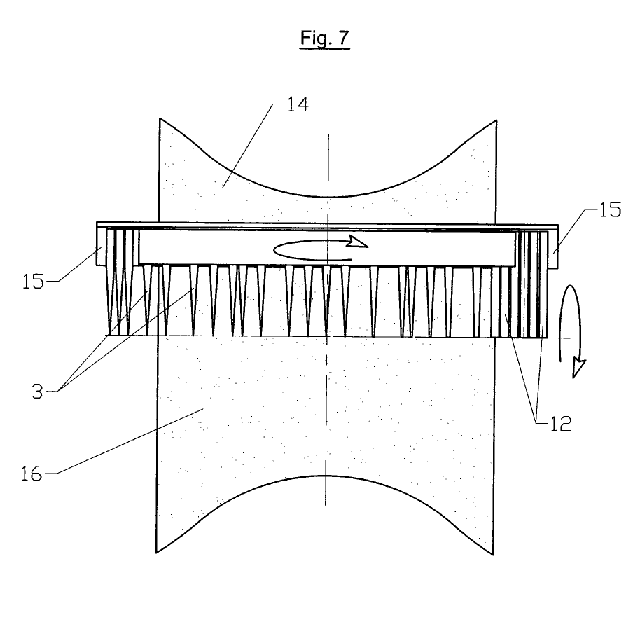

The FIGS. 5, 6, and 7 illustrate schematically an example of application of tapering by means of pointing of cylindrical segments of metallic wires, in order to give them the desired tapered shape before their shaping to the conformation of micromanipulator tips.

According to this method, the cylindrical sections 12 of metallic wires are distributed by a magazine 13 and are drawn one by one, through the intermediary of one of their extreme portions 12a between a drive wheel 14 and a rounded guide path 15. This wheel 14 and/or this guiding path are/is made of a material that presents a good friction coefficient so as to allow moving the sections 12 in rotation around themselves, from the entrance to the exit (arrow F) of the guiding path 15.

During this passage, the portion of the tips that is not taken between the drive wheel 14 and the guiding path 15 is applied against a grinding wheel in the shape of a dual-conical roll. The radius of curvature of the roll is identical to the radius of curvature of the guiding path 15 and the radius of the drive wheel. A portion thus finding itself progressively tapered during the move of the tips from one end to the other of the rounded guiding path 15.

One observes that the axis of rotation of the grinding wheel in the shape of a dual-conical roll 16 is perpendicular to the length of the tips 12-3 (contrary to the cylindrical grinding). It is thus easy, by microscopic observation of the direction of the microgrooves which appear on the surface of the tips, to distinguish the latter from the tips obtained by a traditional method of cylindrical grinding.

At the completion of this grinding the portion 9-6 has overall the shape of a truncated cone.

The flattened part 8 is then formed by stamping in the segment 7 which is then polished by vibrations. After that the elbow 4 is formed by bending the conical portion 11 in one location, and then the finished tip 3 is degreased by ultrasound.

The handle 2 is molded on to the proximal section 7 the flattened part 8 of which has the function of immobilizing it particularly in rotation in relation to this handle 2, once the handle has hardened.

The cost of the tip 3 is especially low. The same is true for the cost of the micromanipulator 1 which can be offered as a disposable item after its first-time use because of the reduced costs. Additionally, the one-time use is already an advantage in itself.

It appears from the preceding that in conformity with the objective of the invention, the cost of the micromanipulator 1 is particularly low, whereas it possesses properties that make it suitable for its use as an ophthalmic surgical micromanipulator.

Such a utilization is illustrated in FIG. 1 where the finger 6 passes into an incision I in the order of 0.5 mm made through the cornea C of an eye E and manipulates a crystalline (not shown for the sake of clarity) in the interior of this eye E during a cataract operation. The particular dimensional characteristics of the tip 3 are such that the finger 6 is perfectly capable of fulfilling its function as a finger for the manipulation of a crystalline in the interior of an eye. In particular the tip 3 presents the rigidity required for the manipulation of the crystalline while its finger 6 is able to act through the incision I and that its free end 10 is capable of efficiently manipulating a crystalline.

FIGS. 3 and 4 show a tip 103 as a variant application of the invention. Like tip 3, this point 103 is intended to be part of an ophthalmic surgical micromanipulator and it features a proximal section 107 meant to be immobilized in the handle or analog of this micromanipulator. The following describes only what distinguishes it from the tip 3. Also, a reference used hereafter to designate a part of the tip 103 analog or equivalent to a referenced part of the tip 3 has been constructed by increasing by 100 the reference identifying this part on the tip 3.

Like section 11, section 111 is made by means of pointing and then by bending in one location. Unlike section 11, section 111 has a diameter dm which decreases only at a single constant rate of linear diminution over its entire length. However, section 11 could also feature several rates of linear diminution, each constant on one of several subsequent portions, like section 11.

The manipulation finger 106 is not straight but has an elbow 120 essentially at a right angle, at a short distance from its free end 110 so as to end by an offset tip 121.

The length L12I of this offset tip 121 is advantageously comprised between 0.5 mm and 0.9 mm, preferably between 0.6 mm and 0.8 mm, for example in the order of 0.7 mm.

The distance L between elbows 104 and 120 is advantageously comprised between 8 mm and 12 mm, preferably between 9 mm and 11 mm, for example in the order of 10 mm.

At the free end 110, the diameter dm of section 111 is, for instance, in the order of 0.22 mm.

The invention does not limit itself to the ways of carrying out the invention that have been described above. In particular the location where the rate of linear diminution of the diameter changes may not be at the level of elbow 4 but may be offset from there along tip 3.

1. Method for producing a surgical micromanipulator tip, the tip being comprised of a proximal mounting base, at least one first elbow, and a distal manipulation finger, the first elbow connecting to the mounting base and terminating in a free end, the mounting base having at least one distal segment, the first elbow and the manipulation finger together a tip portion, the method of producing comprising the steps of:
forming a tip portion with the first elbow and the manipulation finger;
uniform tapering of said tip portion by a pointing method, said pointing method comprising the steps of:
drawing cylindrical sections of metallic wires, one by one, through an intermediary of one extreme portion thereof between a drive wheel and a rounded guiding path;
drawing said cylindrical sections in rotation around themselves, during their travel, from their entrance to their exit from a guiding path; and
during traverse, applying the portion of the cylindrical sections which is not engaged between the drive wheel and the guiding path against a grinding wheel shaped as a dual-conical roll drawn in rotation around an axis perpendicular to the axis of rotation of said cylindrical sections and the radius of curvature identical to the radius of curvature of the guiding path and of the radius of the drive wheel; and
progressively elongating said tip portion during the displacement of the tips from one end to the other of the rounded guiding path.

2. Surgical micromanipulator tip, comprising:
a proximal mounting base, having at least one distal segment;
at least one first elbow connecting to the mounting base and terminating in a free end; and
a distal manipulation finger, said at least one distal segment, the first elbow and the manipulation finger forming a tip portion together, said tip portion tapering in an essentially uniform manner over an entire length thereof in a direction of said free end, and having a diameter decreasing at a rate of essentially constant linear diminution, wherein a succession is presented without detachment, the mounting base, the first elbow and the finger tapering in an essentially uniform manner over respective entire lengths thereof in the direction of said free end, and wherein a succession of at least two truncated cones is presented, the cones being comprised of a first truncated cone and a second truncated cone, said first elbow connecting a proximal basis to a distal top of the first truncated cone, said tip portion being slimmed by pointing.

3. Surgical micromanipulator tip according to claim 2, wherein the rate of linear diminution of the tapering tip portion is between 0.015 mm and 0.025 mm per millimeter of length traveled towards said free end.

4. Surgical micromanipulator tip according to claim 3, wherein manipulation finger presents a length between 8 mm and 13 mm, a diameter between 0.48 mm and 0.60 mm at a level of the first elbow and a diameter between 0.18 mm and 0.42 mm at a level of said free end.

5. Surgical micromanipulator tip according to claim 2, wherein the manipulation finger comprises a second elbow.

6. Surgical micromanipulator tip according to claim 1, wherein the mounting base features a lateral flattened part for immobilization inside a polymer of a handle.

7. Surgical micromanipulator comprising:
a tip according to claim 2; and
a handle housing said tip and being comprised of duplicate mold polymer on at least one section of the mounting base forming the tip.
